(19) United States
(12) Patent Application Publication
(10) Pub. No.: US 2006/0034803 A1
(43) Pub. Date: Feb. 16, 2006

(54) Therapeutic device for osteogenesis

(76) Inventors: Masanori Nakasu, Tokyo (JP); Toshio Matsumoto, Tokyo (JP); Ichoro Ono, Hokkaido (JP)

Correspondence Address: GREENBLUM & BERNSTEIN, P.L.C., 1950 ROLAND CLARKE PLACE, RESTON, VA, 20191, US

(21) Appl. No.: 10/534,360

(22) PCT Filed: Nov. 7, 2003
(86) PCT No.: PCT/JP03/14174

§ 371 Date: May 9, 2005

(30) Foreign Application Priority Data
Nov. 7, 2002 (JP) 2002-324371
Dec. 6, 2002 (JP) 2002-356079

(51) Int. Cl. A61K 48/00

(52) U.S. Cl. 424/93.2; 514/44

(57) ABSTRACT

The object of the present invention is to provide an osteogenic treatment device having excellent bone formation ability and being capable of forming bone having a shape corresponding to a shape of site of implantation. The osteogenic treatment device of the present invention includes nucleic acid containing a base sequence coding for bone morphogenetic protein (BMP), an angiogenesis factor, and a biocompatible base body, and it is to be implanted into a living body for osteogenic treatment. The bone morphogenetic protein contains at least one of BMP-2, BMP-4 and BMP-7, and the nucleic acid contains a base sequence derived from an expression plasmid. The angiogenesis factor is at least one of basic Fibroblast Growth Factor (bFGF), Vascular Endothelial Growth Factor (VEGF), and Hepatocyte Growth Factor (HGF). The base body is formed into a block body formed of a porous material having a porosity of 30 to 95%. Preferably, the base body is mainly formed of hydroxyapatite or tricalcium phosphate. Further, it is preferred that the osteogenic treatment device contains a non-viral vector for holding the nucleic acid. The vector is preferably a cationic liposome. Furthermore, in the osteogenic treatment device of the present invention, it is preferred that when the area (average) of boundary parts between the holes adjacent to each other in the base body is defined as A (μm²) and the maximum cross-sectional area (average) of the holes is defined as B (μm²), the value of B/A is in the range of 2 to 150. Preferably, such an osteogenic treatment device contains either one or both of the vector holding the nucleic acid and the angiogenesis factor.

### FIELD OF THE INVENTION

The present invention relates to an osteogenic treatment device.

### BACKGROUND OF THE INVENTION

In the medical field, attentions are paid to researches on so-called artificial bone, artificial skin, artificial organs and the like. Also at a clinical site, various challenges are made on these technologies from a variety of standpoints. As a result, these technologies have been greatly advanced in the past several years.

This is involved with the fact that allogeneic transplantation has problems that demand continues to far outstrip supply and that there is a risk of unknown infectious diseases.

In order to resolve such problems, it is desired that artificial tissue or artificial organs be developed as a substitute for allograft. In this regard, the same is true of bone transplantation.

The bone formation process and the bone repair process have been made clear by many researchers. As a result, osteoinductive factors that play an important role in controlling the formation or repair of bone have been discovered, identified and isolated. In addition, it has become possible to produce such osteoinductive factors by genetic engineering technique. Further, knowledge about the mechanism of action of the osteoinductive factors has been also accumulated.

Among such osteoinductive factors, many researchers pay their attentions to Bone Morphogenetic Proteins (BMPs) belonging to the superfamily of Transforming Growth Factor b (TGFb). BMPs are active proteins which act on undifferentiated mesenchymal cells in subcutaneous tissue or muscular tissue to differentiate them into osteoblasts or chondroblasts to form bone or cartilage. A basic study has been started to investigate such action of BMPs.

For example, Translated National Publication of Patent Application No. 2001-505097 discloses an implant material which is formed from a matrix material (a base body) on which BMP per se or DNA coding for BMP are carried (held).

However, it has become apparent that such an implant material needs to hold BMP in large quantity, because BMP is protein and therefore it is likely to be inactive. Further, the implant material holding only DNA coding for BMP also has a problem that bone formation does not efficiently occur. The reason for this is supposed as follows. The use of DNA coding for BMP makes it possible to satisfactorily produce BMP, thereby promoting differentiation of undifferentiated mesenchymal cells into osteoblasts or chondroblasts. However, these differentiated cells cannot efficiently grow, and therefore bone formation is not promoted.

Such a matrix material is usually formed so as to have a shape corresponding to the shape of site where the implant material is to be placed, such as a bone defect site. At the site of implantation, the matrix material acts as a field where bone formation occurs.

From the viewpoint of rapid completion of osteogenic treatment, it is preferred that bone formation preferentially occur in the inside of the matrix material.

However, in the case of such a conventional implant material, it is hard to say that bone formation preferentially occurs in the inside of the matrix material. That is, there is a case where bone formation preferentially occurs on the outer surface of the matrix material depending on the internal shape (the shape of holes) of the matrix material used. In such a case, new bone grows far beyond the dimensions of site of implantation, which is disadvantageous to osteogenic treatment.

### SUMMARY OF THE INVENTION

The present invention has been made in view of the problems described above. Accordingly, a main object of the present invention is to provide an osteogenic treatment device having an excellent bone formation ability.

Another object of the present invention is to provide an osteogenic treatment device having an excellent bone formation ability and enabling bone formation along the shape of site of implantation.

In order to achieve these objects, the present invention is directed to an osteogenic treatment device, which comprises: nucleic acid containing a base sequence coding for bone morphogenetic protein (BMP) and a base sequence derived from an expression plasmid; an angiogenesis factor; a non-viral vector for holding the nucleic acid; and a biocompatible base body, wherein the angiogenesis factor is mixed with the nucleic acid, in which the mixing ratio between the angiogenesis factor and the nucleic acid is in the range of about 10:1 to 1:100 by weight.

According to the invention described above, since the osteogenic treatment device contains the nucleic acid containing the base sequence coding for bone morphogenetic protein (BMP) and the base sequence derived from the expression plasmid and the angiogenesis factor, blood vessels which supply various substrates necessary for the growth of osteoblasts are formed around the osteoblasts or chondroblasts (hereinafter, they are representatively referred to as “osteoblasts”) which have been differentiated from undifferentiated mesenchymal cells at the same time or prior to the differentiation into the osteoblasts. Therefore, the various substrates necessary for the growth of osteoblasts are efficiently supplied to the osteoblasts through the blood vessels, and this promotes the growth of osteoblasts. Further, another effect that the angiogenesis factor itself directly acts on the osteoblasts to promote its growth can also be expected.

In particular, in the present invention, the angiogenesis factor is mixed with the nucleic acid so that the mixing ratio between the angiogenesis factor and the nucleic acid is in the range of about 10:1 to 1:100 by weight. This makes it possible that the blood vessels are formed prior to the differentiation of undifferentiated mesenchymal cells into the osteoblasts, and as a result, the above effect is conspicuously exhibited.

Further, since the osteogenic treatment device of the present invention contains the non-viral vector for holding the nucleic acid, the efficiency of the uptake of the nucleic acid into the cells involved in bone formation (such as undifferentiated mesenchymal cells, inflammatory cells and fibroblasts) is adjusted so that the formation of the blood vessels is preferentially made prior to the differentiation of undifferentiated mesenchymal cells into the osteoblasts. With this result, the growth of the osteoblasts is made more efficiently.

These effects are preferably exhibited when using a non-viral vector rather than a vector which is derived from a virus, since such a non-virus vector has a smaller introduction rate of nucleic acid into cells. Namely, when a vector derived from a virus is used, it is possible for cells involved in bone formation to uptake nucleic acid rapidly, and this enables the cells involved in born formation to express BMP so that differentiation from undifferentiated mesenchymal cells into osteoblasts is accomplished at an early stage. However, at this stage, the formation of the blood vessels cannot catch up with it, and thus efficient growth of the osteoblasts in a later stage can not be expected.

In the present invention, it is preferred that the base body is constructed from a porous block body having interconnecting holes in which the adjacent holes communicate to each other. This makes it possible to provide an osteogenic treatment device which has an excellent bone formation ability and enables bone formation along the shape of site of implantation.

In this case, it is preferred that in a case where the area (average) of boundary parts between the holes adjacent to each other in the base body is defined as A (μm²) and the maximum cross-sectional area (average) of the holes is defined as B (μm²), the value of B/A is in the range of 2 to 150. This makes it possible to provide an osteogenic treatment device which promotes bone formation in the base body and enables bone formation corresponding to the shape of the base body, that is the shape of site of implantation.

Further, in this case, it is also preferred that the maximum cross-sectional area (average) B of the holes is in the range of about 7.9×10³ to 1.1×10⁶ μm². This makes it possible to obtain effective osteoconduction in vivo. Namely, it becomes possible to continuously accomplish bone formation inside the holes of the base body embedded in a bone defect site.

Furthermore, in the present invention, it is also preferred that the porosity of the base body is in the range of 30 to 95%. This makes it easy for cells involved in bone formation such as undifferentiated mesenchymal cells, inflammatory cells and fibroblasts and cells involved in blood vessel formation to enter the inside of the base body to allow the base body to be more desirable bone formation site, while maintaining the mechanical strength of the base body appropriately.

Furthermore, in the present invention, it is preferred that the angiogenesis factor is at least one selected from the group comprising basic Fibroblast Growth Factor (bFGF), Vascular Endothelial Growth Factor (VEGF) and Hepatocyte Growth Factor (HGF). Since these factors have excellent blood vessel formation ability, the obtained osteogenic treatment device can have especially excellent bone formation ability.

Moreover, in the present invention, it is also preferred that the bone morphogenetic protein (BMP) is at least one selected from the group comprising BMP-2, BMP-4 and BMP-7. This is because these BMP-2, BMP-4 and BMP-7 have especially excellent function that induces differentiation from undifferentiated mesenchymal cells into osteoblasts.

Further, in the present invention, it is also preferred that the amount of the nucleic acid to be used is in the range of about 1 to 100 μg per 1 mL of the base body. This makes it possible to promote bone formation more rapidly.

Furthermore, in the present invention, it is also preferred that the non-viral vector includes a liposome. This is because a liposome is made of a component similar to that of a cell membrane so that the liposome is relatively easily and smoothly bound to (fused with) the cell membrane. This makes it possible to increase the efficiency of the uptake of the nucleic acid into the cells involved in bone formation.

In this case, it is preferred that the liposome is a cationic liposome. The use of such a cationic liposome is advantageous for the reduction in time for producing the osteogenic treatment device, because it is not necessary to introduce the nucleic acid into the liposome.

In the present invention, it is also preferred that the mixing ratio between the vector and the nucleic acid is in the range of 1:1 to 20:1 by weight. This makes it possible to sufficiently increase the efficiency of the uptake of the nucleic acid into cells involved in bone formation such as undifferentiated mesenchymal cells, inflammatory cells and fibroblasts, while preventing production cost from being increased and cytotoxicity from being developed.

As described above, in the present invention, it is preferred that the base body is formed into a block body, and that the base body is porous. This makes it possible to prevent the osteogenic treatment device from being scattered from the site of implantation at an early stage and progress the bone formation along the shape of the block body. In addition, such a block body can carry the nucleic acid and the angiogenesis factor more easily and reliably, and cells involved in bone formation such as undifferentiated mesenchymal cells, inflammatory cells and fibroblasts and cells involved in blood vessel formation become easy to enter the inside the base body, thus it is advantageous in bone formation.

In this case, it is preferred that the porosity of the base body is in the range of 30 to 95%. This makes it easy for cells involved in bone formation such as undifferentiated mesenchymal cells, inflammatory cells and fibroblasts and cells involved in blood vessel formation to enter the inside of the base body to allow the base body to be more desirable bone formation site, while maintaining the mechanical strength of the base body appropriately.

Further, in the present invention, it is preferred that the base body is mainly formed of hydroxyapatite or tricalcium phosphate. Since hydroxyapatite or tricalcium phosphate has the same structure as the inorganic main component of bone, they have excellent biocompatibility.

The above described and other objects, structures and advantages of the present invention will be more apparent when the following description of the preferred embodiments is considered taken in conjunction with the appended drawings.

### BRIEF DESCRIPTION OF THE DRAWINGS

FIG. 1 shows one example of a recombinant plasmid.

FIG. 2 is a schematic view which shows a cross section of a base body.

FIG. 3 is a schematic view which shows a cross section of a base body for reference.

FIG. 4 shows an electron micrograph of the outer surface of the hydroxyapatite porous sintered body, magnified 50 times.

FIG. 5 shows an electron micrograph of the outer surface of the hydroxyapatite porous sintered body, magnified 50 times.

### BEST MODE FOR CARRYING OUT THE INVENTION

Hereinbelow, an osteogenic treatment device according to the present invention will be described in detail with reference to first and second embodiments.

The osteogenic treatment device according to the present invention comprises nucleic acid containing a base sequence coding for bone morphogenetic protein (BMP), an angiogenesis factor, and a base body, and is to be implanted in a living body to carry out osteogenic treatment.

In view of the problems described above, the present inventors have intensively investigated. As a result, they have found that providing a path for supplying various substrates necessary for cell construction (cell formation) to cells, that is, early formation of blood vessels around osteoblasts or chondroblasts for inducing and growing these cells is important to efficiently grow osteoblasts or chndroblasts differentiated from undifferentiated mesenchymal cells. This finding has led to the completion of the present invention.

According to the present invention, a synergistic effect obtained by using the nucleic acid containing a base sequence coding for BMP and the angiogenesis factor together promotes the differentiation of undifferentiated mesenchymal cells into osteoblasts or chondroblasts, and this enables efficient growth of the differentiated cells, thereby promoting bone formation.

In addition, it can be expected that the angiogenesis factor per se will directly act on the differentiated cells (stem cells) to grow them.

In this specification, the word “bone formation” means formation of both bone and cartilage induced by differentiation of undifferentiataed mesenchymal cells into osteoblasts and chondroblasts (hereinafter, represented by “osteoblasts”).

Further, the word “osteogenic treatment” means prevention of diseases to be treated by formation or compensation of bone tissue or cartilage tissue, or treatment or improvement of such diseases in the medical and dental fields.

Hereinbelow, a first embodiment of the osteogenic treatment device according to the present invention will be described in detail.

In the following description, nucleic acid containing a base sequence coding for bone morphogenetic protein (BMP) is used as a representative of osteoinductive factors. This is because the use of nucleic acid containing a base sequence coding for BMP as an osteoinductive factor makes it possible to more rapidly promote bone formation.

As a base sequence coding for BMP, cDNA is usually used. Therefore, in the following description, a base sequence coding for BMP is referred to as “BMP cDNA”.

BMP to be used in the present invention is not particularly limited as long as it has activity that induces differentiation of undifferentiated mesenchymal cells into osteoblasts to promote bone formation. Examples of such BMP include BMP-1, BMP-2, BMP-3, BMP-4, BMP-5, BMP-6, BMP-7, BMP-8, BMP-9, and BMP-12 (homodimers), heterodimers thereof, and modified versions thereof (that is, proteins each having an amino acid sequence obtained by subjecting an amino acid sequence of naturally-occurring BMP to deletion, substitution, and/or addition of 1 or more amino acids, while maintaining the same activity as that of the naturally-occurring BMP). Among these BMPs, at least one of BMP-2, BMP-4, and BMP-7 is particularly preferable. This is because BMP-2, BMP-4, and BMP-7 are particularly superior in activity that induces differentiation of undifferentiated mesenchymal cells into osteoblasts, thereby enabling to obtain an osteogenic treatment device having especially high bone formation ability.

For this reason, BMP cDNA to be used in the present invention should have a base sequence capable of producing (expressing) the BMP mentioned above. Examples of such BMP cDNA include a base sequence coding for naturally-occurring BMP and a base sequence obtained by subjecting a base sequence coding for naturally-occurring BMP to deletion, substitution, and/or addition of 1 or more bases. These BMP cDNAs can be used singly or in combination of two or more of them.

Such BMP cDNAs can be obtained by the methods described in, for example, Translated National Publications of Patent Applications Nos. Hei 2-500241, Hei 3-503649, and Hei 3-505098.

Further, the nucleic acid is preferably one containing a base sequence derived from an expression plasmid, that is, one obtained by splicing (introducing) BMP cDNA into an expression plasmid.

Hereinafter, one obtained by splicing BMP cDNA into an expression plasmid is referred to as a “recombinant plasmid”, and the recombinant plasmid will be described as a representative of the nucleic acid containing a base sequence coding for BMP.

Such a recombinant plasmid is taken up by undifferentiated mesenchymal cells, inflammatory cells, or fibroblasts (hereinafter, collectively called “cells involved in bone formation”), thereby extremely increasing the expression efficiency of BMP in these cells.

An expression plasmid to be used can be selected from various plasmids widely used in the field of genetic engineering. Examples of such an expression plasmid include pCAH, pSC101, pBR322, and pUC18, and these plasmids can be used singly or in combination of two or more of them.

Into the recombinant plasmid, a base sequence (a DNA fragment) for properly controlling the expression of BMP may be introduced when necessary.

The BMP cDNA and other various base sequences can be spliced into an expression plasmid by a well-known method.

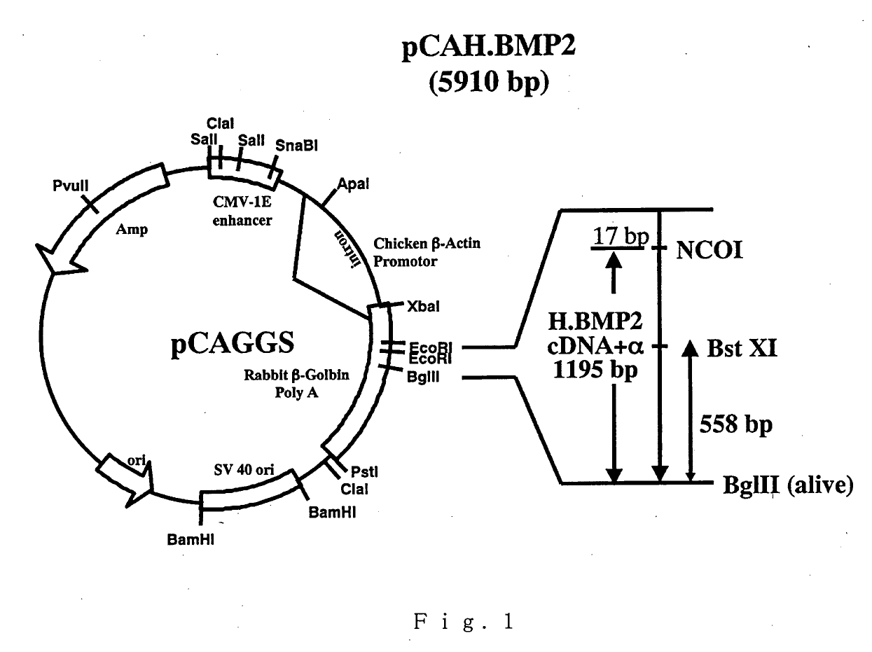

FIG. 1 shows one example of a recombinant plasmid (chimeric DNA), which is obtained by introducing BMP-2 cDNA into pCAH which is an expression plasmid.

This recombinant plasmid contains a DNA fragment resistant to Amp (ampicillin). In addition, a DNA fragment containing an enhancer-promoter derived from cytomegalovirus (CMV) and a DNA fragment containing a transcription termination signal derived from SV40 are spliced into the recombinant plasmid. The DNA fragment containing a transcription termination signal derived from SV40 is located downstream from the BMP-2 cDNA.

The amount of the recombinant plasmid (nucleic acid) to be used is not particularly limited, but is preferably in the range of about 1 to 100 μg, more preferably in the range of about 10 to 70 μg, per 1 mL of the base body (which will be described later). If the amount of the recombinant plasmid to be used is too small, there is a case where it is impossible to rapidly promote bone formation. On the other hand, even if the amount of the recombinant plasmid to be used exceeds the above upper limit value, an effect obtained by using the recombinant plasmid is not enhanced in proportion to the amount thereof.

The angiogenesis factor to be used in the present invention is not particularly limited as long as it can promote the formation of blood vessels. Examples of such an angiogenesis factor include basic Fibroblast Growth Factor (bFGF), Vascular Endothelial Growth Factor (VEGF), Hepatocyte Growth Factor (HGF), Granulocyte Macrophage-Colony Stimulating Factor (GM-CSF), Granulocyte-Colony Stimulating Factor (G-CSF), Macrophage-Colony Stimulating Factor (M-CSF), Stem Cell Factor (SCF), Angiopoietin-1, Angiopoietin-2, ribonuclease-like protein, nicotinamide, prostaglandin E (prostaglandin E₁, prostaglandin E₂, prostaglandin E₃), proline derivatives, and cyclic AMP derivatives such as dibutyl cyclic AMP (dBc AMP). These angiogenesis factors can be used singly or in combination of two or more of them. Among them, at least one of basic Fibroblast Growth Factor (bFGF), Vascular Endothelial Growth Factor (VEGF), and Hepatocyte Growth Factor (HGF) is particularly preferable. This is because they are superior in blood vessel formation ability, thereby enabling to obtain an osteogenic treatment device having especially high bone formation ability.

The amount of the angiogenesis factor to be used is appropriately determined depending on the kind thereof, and is not particularly limited. The mixing ratio between the angiogenesis factor and the recombinant plasmid (nucleic acid) is preferably in the range of about 10:1 to 1:100 by weight, more preferably in the range of about 1:1 to 1:100 by weight. If the amount of the angiogenesis factor to be used is too small, there is a case where new blood vessels are not efficiently formed depending on the kind of angiogenesis factor used so that osteoblasts do not sufficiently grow. On the other hand, even if the angiogenesis factor is used in an amount exceeding the upper limit of the ratio described above, an effect obtained by using the angiogenesis factor is not enhanced in proportion to the amount thereof.

The osteogenic treatment device according to the present invention further comprises a biocompatible base body. The base body acts as a field where new bone is formed by osteoblasts differentiated from undifferentiated mesenchymal cells.

Preferably, such a base body is formed into a block body. This is because a block body (e.g., a sintered body) has shape stability. That is, in a case where an osteogenic treatment device comprised of a block body is implanted in a living body, a phenomenon in which the osteogenic treatment device is scattered outside the site of implantation in an early stage after implantation does not occur. Particularly, in a case where such an osteogenic treatment device is implanted in a relatively large bone defect site, there is an advantage that bone formation occurs along the shape of the block body.

The form of the base body may be appropriately selected depending on a site where the osteogenic treatment device is to be applied (implanted). For example, the base body may be in powder, granule, or pellet (small block) form. In such a case, a composition obtained by mixing the base body, the recombinant plasmid (nucleic acid), and the angiogenesis factor can be used as an osteogenic treatment device, which is used so as to fill a bone defect site.

Further, it is preferred that the base body is porous. By using such a porous base body, it is possible to allow the base body to hold the recombinant plasmid (nucleic acid) and the angiogenesis factor easily and reliably. In addition, it is also possible to allow cells involved in bone formation and cells involved in blood vessel formation (e.g., vascular endothelial cells) to easily enter the inside of the base body. This is advantageous for bone formation.

The porosity of the base body is not particularly limited, but is preferably in the range of about 30 to 95%, more preferably in the range of about 55 to 90%. By setting the porosity of the base body to a value within the above range, it is possible to allow cells involved in bone formation and cells involved in blood vessel formation to enter the inside of the base body more easily while maintaining the mechanical strength of the base body. That is, the base body becomes more suitable for bone formation. The porosity of the base body can be measured, for example, based on the visual image of SEM (Scanning Electron Microscope) or using a porosimeter.

The constituent material of the base body is not particularly limited as long as it is biocompatible. Examples of such a material include hydroxyapatite, fluorapatite, carbonate apatite, calcium phosphate-type compounds such as dicalcium phosphate, tricalcium phosphate, tetracalcium phosphate, and octacalcium phosphate, ceramics such as alumina, titania, zirconia, and yttria, and various metal materials such as titanium, titanium alloys, stainless steel, Co—Cr-based alloys, and Ni—Ti-based alloys. These materials can be used singly or in combination of two or more of them.

Among them, calcium phosphate-type compounds and ceramics such as alumina and zirconia (so-called bioceramics) are preferably used as a constituent material of the base body. Particularly, hydroxyapatite or tricalcium phosphate is preferably used as a main material of the base body.

Hydroxyapatite and tricalcium phosphate have especially excellent biocompatibility because they have the same structure as that of a main inorganic component of bone. Further, in a case where a liposome is used as a vector (this case will be described later in detail), the base body mainly made of hydroxyapatite or tricalcium phosphate can hold the liposome stably for a long time because hydroxyapatite or tricalcium phosphate has both positive and negative charges. As a result, the recombinant plasmid (nucleic acid) adsorbed to or incorporated in the liposome is also carried by the base body stably for a long time, which contributes to more rapid bone formation. Further, hydroxyapatite and tricalcium phosphate are preferable in that they can hold new bone due to their high affinity for osteoblasts.

Such a porous base body can be formed (produced) by various methods. For example, in a case where a porous block body as a base body is formed using ceramics, a slurry containing ceramics powder is molded so as to have a shape corresponding to the shape of the site of implantation such as a bone defect site by, for example, compression molding to obtain a molded body, and then the molded body is sintered. In this way, a porous block body is obtained. Alternatively, a porous block body may be formed by mixing ceramics powder and an aqueous solution of water-soluble polymer under stirring, pouring the mixture into a mold, drying it to obtain a molded body, shaping the molded body into a desired form, and sintering the molded body.

The osteogenic treatment device described above can be formed (produced) by bringing the recombinant plasmid (nucleic acid) and the angiogenesis factor into contact with the base body. Specifically, the osteogenic treatment device can be easily formed by adding a liquid (a solution or a suspension) containing the recombinant plasmid and a liquid (a solution or a suspension) containing the angiogenesis factor to the base body, or by adding a liquid containing both of the recombinant plasmid and the angiogenesis factor to the base body. Alternatively, the osteogenic treatment device may be formed by immersing the base body in a liquid (a solution or a suspension) containing the recombinant plasmid and a liquid (a solution or a suspension) containing the angiogenesis factor or in a liquid containing both of the recombinant plasmid and the angiogenesis factor.

In a case where the base body is in powder, granule or pellet form, the osteogenic treatment device is formed by, for example, mixing the base body, a binder, and the liquid containing the recombinant plasmid and the liquid containing the angiogenesis factor described above or the liquid containing both of the recombinant plasmid and the angiogenesis factor described above, kneading them to obtain a kneaded material, and molding the kneaded material.

When the osteogenic treatment device according to the present invention is placed in (applied to) the site of implantation such as a bone defect site, cells involved in bone formation existing close to the osteogenic treatment device uptake the recombinant plasmid (nucleic acid). In these cells, BMP is successively produced by using the recombinant plasmid as a template, and the BMP induces the differentiation of undifferentiated mesenchymal cells into osteoblasts. At the same time, new blood vessels are actively formed in the base body (i.e., around the osteoblasts) due to the activity of the angiogenesis factor, thereby enabling various substrates necessary for the growth of osteoblasts to be supplied to the osteoblasts through the blood vessels. This makes it possible that the osteoblasts grows efficiently, thereby promoting bone formation.

Further, it is preferred that the osteogenic treatment device contains a vector. This vector has the function of holding the recombinant plasmid (nucleic acid) and promoting the uptake of the recombinant plasmid into cells involved in bone formation. By using such a vector, it is possible to improve the efficiency of the uptake of the recombinant plasmid into the cells involved in bone formation, thereby more rapidly promoting bone formation.

As a vector, either a vector which is not derived from a virus (i.e., a non-viral vector) or a vector which is derived from a virus such as an adenovirus vector or a retrovirus vector may be used, but a non-viral vector is preferably used. By using a non-viral vector, it is possible to supply a circumscribed site with a relatively large amount of the recombinant plasmid easily and reliably. In addition, it is also possible to provide a high level of safety for patients because infection does not occur. Further, a method using a non-viral vector is excellent in that it is possible to save time and effort. This is because an ex-vivo method using a virus vector or a cell requires, for example, the operation to introduce nucleic acid into a virus vector or a cell and the operation to grow the virus vector or the cell into which nucleic acid has been introduced, but a method using a non-viral vector does not require these operations.

As a non-viral vector, a liposome (a lipid membrane) is preferably used among various non-viral vectors. This is because a liposome is made of a component similar to that of a cell membrane so that the liposome is relatively easily and smoothly bound to (fused with) a cell membrane. This makes it possible to increase the efficiency of the uptake of the recombinant plasmid into the cells involved in bone formation.

As a liposome, for example, a cationic liposome capable of adsorbing the recombinant plasmid to the surface thereof, or an anionic liposome capable of internally incorporating the recombinant plasmid can be used. These liposomes can be used singly or in combination.

A cationic liposome is mainly made of polycationic lipid such as DOSPA (2,3-dioleyloxy-N-[2(sperminecarboxamido)ethyl]-N,N-dimethy l-1-propanaminium trifluoroacetate). As such a cationic liposome, for example, “SuperFect” (QIAGEN) commercially available can be used.

On the other hand, an anionic liposome is mainly made of phospholipid such as 3-sn-phosphatidyl choline, 3-sn-phosphatidyl serine, 3-sn-phosphatidyl ethanolamine, 3-sn-phosphatidal ethanolamine, or derivatives thereof.

Further, the liposome to be used in the present invention may contain an additive for stabilizing a lipid membrane, such as cholesterol.

In the present invention, a cationic liposome is preferably used. The use of a cationic liposome is advantageous for the reduction in time for producing the osteogenic treatment device, because it is not necessary to introduce the recombinant plasmid into the liposome.

The amount of the vector to be used is appropriately determined depending on the kind thereof, and is not particularly limited. The mixing ratio between the vector and the recombinant plasmid (nucleic acid) is preferably in the range of about 1:1 to 20:1 by weight, more preferably in the range of about 2:1 to 10:1 by weight. If the amount of the vector to be used is too small, there is a case where it is impossible to sufficiently improve the efficiency of the uptake of the recombinant plasmid into the cells involved in bone formation. On the other hand, even if the vector is used in an amount exceeding the upper limit of the ratio described above, an effect obtained by using the vector is not enhanced in proportion to the amount thereof. In addition, the use of such an excessive amount of vector increases production costs and has a possibility that cytotoxicity develops, which is disadvantageous for osteogenic treatment.

Although the first embodiment of the osteogenic treatment device according to the present invention has been described above, the present invention is not limited thereto.

In the first embodiment, a recombinant plasmid obtained by splicing BMP cDNA into an expression plasmid has been described as a representative of nucleic acid containing a base sequence coding for BMP, but nucleic acid containing a base sequence coding for BMP to be used in the present invention may be, for example, BMP cDNA (not spliced into an expression plasmid), mRNA of BMP, or one obtained by arbitrarily adding a base(s) to the BMP cDNA or the mRNA of BMP.

### EXAMPLES

Hereinbelow, actual examples of the first embodiment of the present invention will be described.

### Example 1A

1. Preparation of Recombinant Plasmid

A recombinant plasmid shown in FIG. 1 was obtained by splicing human BMP-2 cDNA (a base sequence coding for human BMP-2) and desired base sequences into an expression plasmid according to a well-known method.

Then, the thus obtained recombinant plasmid was grown in the following manner.

First, the recombinant plasmid was added to 200 μL of a suspension of DH5α (Competent Bacteria) at room temperature to obtain a mixture.

Next, the mixture was added to LB agar medium to carry out cultivation for 12 hours at 37° C.

After the completion of the cultivation, a relatively large colony was selected from colonies grown on the LB agar medium, and was inoculated onto LB agar medium containing Amp (ampicillin) to carry out cultivation for 12 hours at 37° C.

After the completion of the cultivation, the cell membrane of DH5α grown on the LB agar medium containing Amp was broken to obtain a solution, and the recombinant plasmid was purified and separated from the solution.

2. Production of Hydroxyapatite Porous Sintered Body (Base Body)

Hydroxyapatite was synthesized according to a well-known wet synthesis method to obtain a slurry of hydroxyapatite.

The slurry of hydroxyapatite was spray-dried to obtain hydroxyapatite powder having an average particle size of about 15 μm. Thereafter, the hydroxyapatite powder was calcined for 2 hours at 700° C., and was then pulverized by a pulverizer usually used to obtain hydroxyapatite powder having an average particle size of about 12 μm. The hydroxyapatite powder and an aqueous solution of water-soluble polymer were mixed and were then stirred to foam the mixture. In this way, a paste of the mixture was obtained. It is to be noted that the mixing ratio between the hydroxyapatite powder and the aqueous solution of water-soluble polymer was 5:6 by weight.

The paste of the mixture was poured into a mold, and was then dried at 80° C. so that the water-soluble polymer was turned into a gel to obtain a molded body. The molded body was machined so as to have a disk-like shape with a diameter of 10 mm and a thickness of 3 mm (volume: about 0.24 mL) by the use of a processing machine such as a lathe usually used.

The disk-like molded body was sintered for 2 hours at 1,200° C. in the atmosphere to obtain a hydroxyapatite porous sintered body.

The porosity of the hydroxyapatite porous sintered body was 70%. The porosity was measured by the Archimedean method.

3. Production of Osteogenic Treatment Device

A phosphate buffer containing the recombinant plasmid, a phosphate buffer containing basic Fibroblast Growth Factor (bFGF) as an angiogenesis factor, and a phosphate buffer containing a cationic liposome (“SuperFect”, QIAGEN) as a vector were prepared. The hydroxyapatite porous sintered body was impregnated with these phosphate buffers so as to contain 10 μg of the recombinant plasmid, 1 μg of basic Fibroblast Growth Factor (bFGF), and 40 μg of the cationic liposome. In this way, an osteogenic treatment device was obtained.

### Comparative Example 1A

An osteogenic treatment device was produced in the same manner as in Example 1A except that basic Fibroblast Growth Factor was not used.

### Comparative Example 2A

An osteogenic treatment device was produced in the same manner as in Example 1A except that the recombinant plasmid and the cationic liposome were not used.

### Comparative Example 3A

An osteogenic treatment device was produced in the same manner as in Example 1A except that the recombinant plasmid and the basic Fibroblast Growth Factor were not used.

Evaluation

1. Evaluation Test

72 domestic rabbits (average body weight: 3.0 kg) were prepared. An operation was performed on each of the domestic rabbits in the following manner.

First, each domestic rabbit was anesthetized with an intravenous injection of 25 mg/kg pentobarbital sodium (“Nembutal”, Abbot Laboratories).

Next, an incision was made in the scalp of the domestic rabbit, and then a flap (width: 2.5 cm, length: 3.0 cm) with a pedicle at the caudal end was elevated.

Next, a 2 to 3 mm of incision was made in the exposed periosteum, and the periosteum having a diameter of about 3 mm was separated from the skull using a periosteum elevator to expose the skull.

Next, a craniotome was set near the midline of the exposed skull, and then a through hole was made in the skull by removing it in such a manner that the dura mater was left intact. Thereafter, bleeding was completely stopped. The thickness of the skull was about 3 mm, and the hole made in the skull had a diameter of about 1.2 cm.

Next, the 72 domestic rabbits subjected to craniotomy were divided into 4 groups each containing 18 rabbits. The osteogenic treatment devices of Example 1A, Comparative Example 1A, Comparative Example 2A, and Comparative Example 3A were implanted in the domestic rabbits of group 1, group 2, group 3, and group 4, respectively. After implantation, in each of the rabbits, the flap was returned to the initial position and then the scalp incision was sutured.

Each of the domestic rabbits subjected to such an operation was bred in an individual cage.

2. Evaluation Result

The six domestic rabbits of each of the groups were killed after the lapse of 3, 6, and 9 weeks from the operation with an overdose of the anesthetic described above.

A piece of the skull and skin on the skull were together removed from each domestic rabbit to sample tissue. The tissue was immediately immersed in 10% neutral buffered formalin for fixation, and was then embedded in polyester resin. The tissue embedded in polyester resin was cut and polished to obtain a slice of the tissue having a thickness of 50 μm. Then, cole-HE staining was carried out to obtain a specimen of the tissue.

A bone formation rate was determined using the tissue specimen in the following manner.

Specifically, an image of the tissue specimen was taken with a stereoscopic microscope system SZX-12 provided with a digital camera (DP-12) (Olympus). Next, data of a portion where new bone had been formed was extracted from the image data by digital processing using Photoshop_ver 4.0 (Adobe). Then, the area of the portion where new bone had been formed was measured and digitized by an image analysis method using Scion Image (Scion) to determine a bone formation rate.

In this regard, it is to be noted that the bone formation rate was determined in an area of 5 mm (measured from the end of the hydroxyapatite porous sintered body in the surface direction thereof, that is, in a direction perpendicular to the thickness direction)×3 mm (that is, the thickness of the hydroxyapatite porous sintered body).

The results are shown in Table 1.

TABLE 1

| | | Evaluation Results | | |
|---|---|---|---|---|
| | | Bone Formation Rate (%) | | |
| | Osteogenic Treatment Device | 3 weeks | 6 weeks | 9 weeks |
| Example 1A | Recombinant Plasmid Basic Fibroblast Growth Factor | 5.3 ± 2.2 | 18.0 ± 9.7 | 42.0 ± 10.5 |
| | Cationic Liposome HAp Porous Sintered Body | | | |
| Com. Ex. 1A | Recombinant Plasmid Cationic Liposome | 14.1 ± 7.5 | 18.5 ± 4.9 | 29.2 ± 10.1 |
| | HAp Porous Sintered Body | | | |
| Com. Ex. 2A | Basic Fibroblast Growth Factor | 3.2 ± 3.2 | 19.5 ± 1.4 | 31.0 ± 9.6 |
| | HAp Porous Sintered Body | | | |
| Com. Ex. 3A | Cationic Liposome | 7.7 ± 3.4 | 9.0 ± 8.0 | 12.7 ± 7.6 |
| | HAp Porous Sintered Body | | | |

HAp: Hydroxyapatite

As shown in Table 1, the bone formation rate of the osteogenic treatment device of Example 1A (that is, the osteogenic treatment device according to the present invention) was equal to or lower than that of each of the osteogenic treatment devices of Comparative Examples 1A to 3A after a lapse of 3 or 6 weeks, but became significantly higher after a lapse of 9 weeks as compared to any other osteogenic treatment devices of Comparative Examples.

From the result, it has become apparent that using the recombinant plasmid and basic Fibroblast Growth Factor (bFGF) together makes it possible to obtain an osteogenic treatment device having extremely excellent bone formation ability.

Further, osteogenic treatment devices were produced in the same manner as in Example 1A and Comparative Examples 1A to 3A, respectively except that in each of the devices a tricalcium phosphate porous sintered body was used as a base body instead of a hydroxyapatite porous sintered body, to carry out the same evaluation test as that described above. The evaluation results were substantially the same as those of Example 1A and Comparative Examples 1A to 3A described above.

Furthermore, osteogenic treatment devices were produced in the same manner as in Example 1A except that a base sequence coding for human BMP-2 was changed to a base sequence coding for any one of human BMP-1, human BMP-3, human BMP-4, human BMP-5, human BMP-6, human BMP-7, human BMP-8, human BMP-9, and human BMP-12 or these base sequences coding for human BMP-1 to 9 and 12 were arbitrarily combined, to carry out the same evaluation test as that described above. The evaluation results were substantially the same as that of Example 1A.

Moreover, osteogenic treatment devices were produced in the same manner as in Example 1A except that the angiogenesis factor was changed from basic Fibroblast Growth Factor (bFGF) to Vascular Endothelial Growth Factor (VEGF) or Hepatocyte Growth Factor (HGF) or these angiogenesis factors were arbitrarily combined, to carry out the same evaluation test as that described above. The evaluation results were substantially the same as those of Example 1A.

From these results, it can be said that the use of the osteogenic treatment device according to the first embodiment of the present invention enables very rapid bone formation, which contributes to rapid osteogenic treatment.

Particularly, in the present invention, since the angiogenesis factor is used, new blood vessels are actively formed around osteoblasts. This enables efficient growth of osteoblasts and thereby rapid bone formation.

Therefore, the osteogenic treatment device according to the present invention eliminates a necessity to harvest bone to carry out free bone graft. This makes it possible to rationally perform an operation for osteogenic treatment with safety and reliability.

In addition, it is also possible to reduce the time required for an operation, the duration of hospitalization, and total medical costs and to improve the quality of treatment and the QOL of patients.

Further, the use of the vector holding the nucleic acid enhances the efficiency of the uptake of the nucleic acid into the cells involved in bone formation, such as undifferentiated mesenchymal cells, inflammatory cells, and fibroblasts. This enables more rapid bone formation, thereby further enhancing the above-described effects obtained by using the osteogenic treatment device according to the present invention.

Furthermore, the use of the block body as a base body makes it possible to promote bone formation along the shape of the base body. This is advantageous to treat a relatively large bone defect site.

Moreover, the use of the porous base body which is specific to gene therapy makes it possible to allow the base body to hold the nucleic acid and the angiogenesis factor more easily and reliably. In addition, it becomes easy for various cells involved in bone formation and cells involved in blood vessel formation to enter the inside of the base body. This is also advantageous for bone formation.

Moreover, the osteogenic treatment device according to the present invention can be easily stored, handled, and shaped into a desired form during an operation.

Hereinbelow, a second embodiment of the present invention will be described.

The osteogenic treatment device according to the present invention has a base body comprised of a porous block body (e.g., a sintered body). The base body acts as a field where new bone is formed by osteoblasts differentiated from undifferentiated mesenchymal cells.

In such a porous block body, each hole is not a closed hole separated from adjacent holes. That is, the porous block body has interconnecting holes.

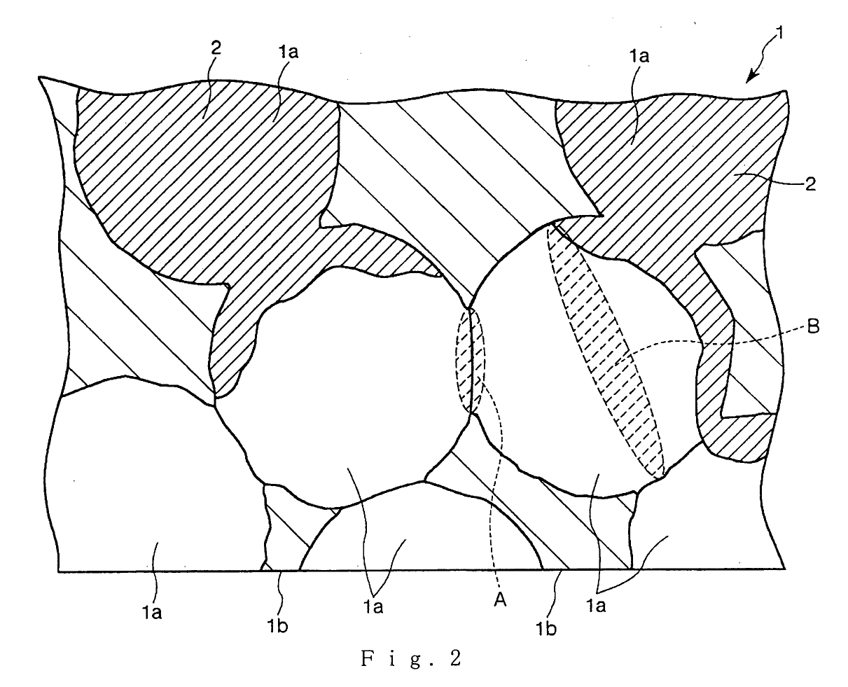

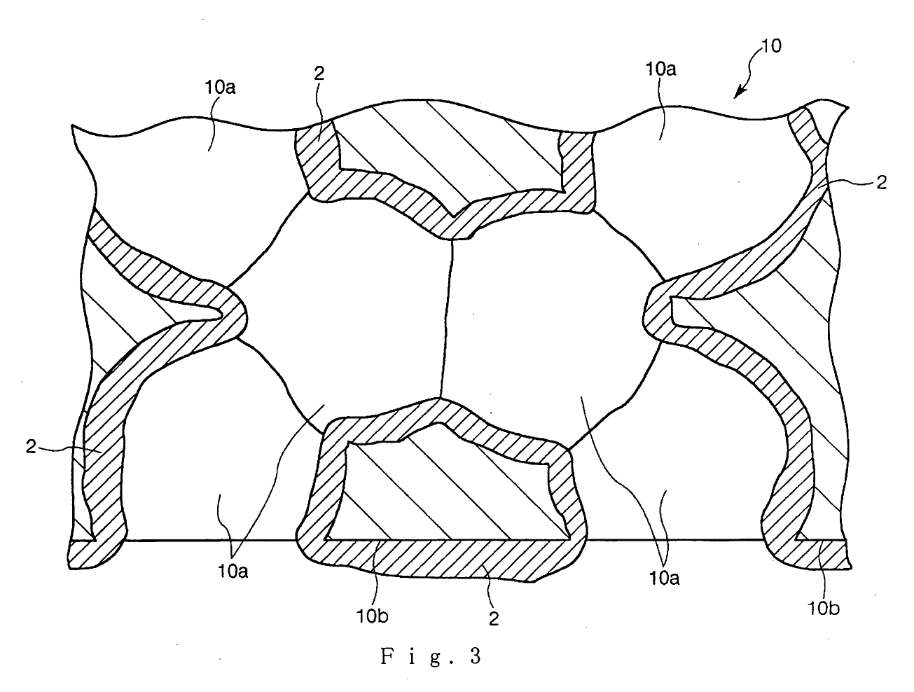

FIG. 2 is a schematic view which shows a cross section of the base body of the second embodiment of the present invention, and FIG. 3 is a schematic view which shows a cross section of a base body for reference. It is to be noted that a reference numeral “2” in FIGS. 2 and 3 represents new bone.

The feature of the osteogenic treatment device of the second embodiment of the present invention is that the area (average) of boundary parts between holes 1a and 1a adjacent to each other in a base body 1 is appropriately determined with respect to the maximum cross-sectional area (average) of the holes. Specifically, in a case where the area (average) of boundary parts between the holes 1a and 1a adjacent to each other in the base body 1 is defined as A (μm²) and the maximum cross-sectional area (average) of the holes is defined as B (μm²) as shown in FIG. 2, the value of B/A is in the range of 2 to 150, preferably in the range of 2.5 to 125, more preferably in the range of 3.0 to 100.

In a case where the value of B/A is less than the above lower limit value, that is, in the case of a base body 10 shown in FIG. 3 in which the area (average) of boundary parts between holes 10a and 10a adjacent to each other is relatively large, cells involved in bone formation smoothly move along the inner surface of the base body 10 from a hole 10a to an adjacent hole 10a to reach the outer surface 10b of the base body 10. As a result, new bone 2 is formed in the inside of the base body 10 and on the outer surface 10b of the base body 10 at substantially the same time, or new bone 2 is preferentially formed on the outer surface 10b of the base body 10.

On the other hand, in a case where the value of B/A exceeds the above upper limit value, that is, in a case where the area (average) of boundary parts between holes adjacent to each other is very small (not shown), cells involved in bone formation are difficult to enter the inside of the base body. As a result, new bone is preferentially formed on the outer surface of the base body.

However, in the present invention, since the value of B/A is set to a value within the above range, cells involved in bone formation move (diffuse) in the base body 1 in such a manner that they fill a hole 1a first and then overflow into an adjacent hole 1a. As a result, new bone 2 is formed in the inside of the base body 1 as shown in FIG. 2. Therefore, the use of the osteogenic treatment device according to the present invention enables bone formation along the shape of the base body 1, that is, along the shape of site of implantation such as a bone defect site. This contributes to rapid osteogenic treatment.

Specifically, the maximum cross-sectional area (average) B of the holes is preferably in the range of about 7.9×10³ to 1.1×10⁶ μm², more preferably in the range of about 1.8×10⁴ to 7.9×10⁵ μm². These values correspond to 100 to 1,200 μm (preferably, 150 to 1,000 μm) as expressed in terms of an average pore size. By setting the maximum cross-sectional area (average) B of the hole to a value within the above range, it is possible to prevent the base body from inhibiting rapid bone formation induced by BMP, thereby enabling new bone to be formed in the holes.

The porosity of the base body is not particularly limited, but is preferably in the range of about 30 to 95%, more preferably in the range of about 55 to 90%. By setting the porosity of the base body to a value within the above range, the base body becomes more suitable for bone formation while maintaining good mechanical strength. It is to be noted that the porosity of the base body can be measured, for example, based on a visual image of SEM (Scanning Electron Microscope) or using a porosimeter.

As is the case with the first embodiment, the constituent material of the base body is not particularly limited as long as it is biocompatible. Examples of such a material include hydroxyapatite, fluorapatite, carbonate apatite, calcium phosphate-type compounds such as dicalcium phosphate, tricalcium phosphate, tetracalcium phosphate, and octacalcium phosphate, ceramics such as alumina, titania, zirconia, and yttria, and various metal materials such as titanium, titanium alloys, stainless steel, Co—Cr-based alloys, and Ni—Ti-based alloys. These materials can be used singly or in combination of two or more of them.

Among them, calcium phosphate-type compounds and ceramics such as alumina and zirconia (so-called bioceramics) are preferably used as a constituent material of the base body. Particularly, hydroxyapatite or tricalcium phosphate is preferably used as a main material of the base body.

Hydroxyapatite and tricalcium phosphate have especially excellent biocompatibility because they have the same (similar) composition, structure, and properties as those of a main inorganic component of bone. Particularly, in a case where a liposome is used as a vector, the base body mainly made of hydroxyapatite or tricalcium phosphate can hold the liposome stably for a long time because hydroxyapatite or tricalcium phosphate has both positive and negative charges. As a result, a recombinant plasmid (nucleic acid) adsorbed to or incorporated in the liposome is also carried by the base body stably for a long time, which contributes to more rapid bone formation. Further, hydroxyapatite and tricalcium phosphate are preferable in that they can hold new bone due to their high affinity for osteoblasts.

Such a base body can be formed (produced) by various methods. For example, a porous block body as a base body made of ceramics is formed in the following manner. A slurry of ceramics containing a water-soluble polymer is foamed by stirring, and then the slurry is formed into a block shape and dried to obtain a porous block body. The block body is machined by, for example, a processing machine usually used such as machining center so as to have a shape corresponding to the shape of the site of implantation such as a bone defect site, and is then sintered.

A desired value of B/A can be obtained by appropriately setting, for example, conditions for synthesizing raw material powder (e.g., primary particle size, degree of dispersion of primary particles), conditions of raw material powder (e.g., average particle size, whether calcining is carried out or not, whether pulverizing is carried out or not), conditions for foaming a slurry by stirring (e.g., kind of surfactant to be used, degree of power for stirring a slurry), or sintering conditions (e.g., sintering atmosphere, sintering temperature) in the base body forming method. For example, in a case where a sintering temperature is made relatively high, the diffusion of the raw material powder is promoted. As a result, the value of A tends to become small, that is, the value of B/A tends to become large.

Particularly, since the conditions of raw material powder and the conditions for foaming a slurry by stirring have a great influence on the value of B/A, it is preferred that these conditions be closely controlled.

It is to be noted that various conditions for obtaining a desired value of B/A can be experimentally determined.

The osteogenic treatment device described above can be formed (produced) by bringing a recombinant plasmid (nucleic acid) into contact with the base body. Specifically, the osteogenic treatment device can be easily formed by, for example, adding a liquid (a solution or a suspension) containing a recombinant plasmid to the base body. Alternatively, the osteogenic treatment device may be formed by immersing the base body in a liquid (a solution or a suspension) containing the recombinant plasmid.

In a case where a base body precursor is in powder, granule, or pellet form, the osteogenic treatment device may be formed by, for example, mixing the base body precursor, a binder, and the above-described liquid containing the recombinant plasmid, kneading them to obtain a kneaded material, and molding the kneaded material.

When the osteogenic treatment device according to the present invention is placed in (applied to) the site of implantation such as a bone defect site, cells involved in bone formation existing close to the osteogenic treatment device take up the recombinant plasmid (nucleic acid). In these cells, BMP is successively produced by using the recombinant plasmid as a template, and the BMP induces the differentiation of undifferentiated mesenchymal cells into osteoblasts. As a result, bone formation occurs. It is to be noted that bone formation is rapidly promoted in a direction from the outer surface to the inside of the base body along the shape of the base body (along the shape the of site of implantation).

Hereinbelow, a vector and an angiogenesis factor will be described. The vector and the angiogenesis factor to be used in the second embodiment are the same as those of the first embodiment (for more details about them, see the above description of the first embodiment).

As is the case with the first embodiment, a vector to be used in the second embodiment has the function of holding a recombinant plasmid (nucleic acid) and promoting uptake of the recombinant plasmid into cells involved in bone formation. By using such a vector, it is possible to improve the efficiency of the uptake of the recombinant plasmid into the cells involved in bone formation, thereby more rapidly promoting bone formation. For more details on the vector, see the above description about it.

An angiogenesis factor acts on cells involved in blood vessel formation (e.g., vascular endothelial cells) to promote the formation of new blood vessels. By using such an angiogenesis factor, it is possible to form new blood vessels in the inside (holes) of the base body, that is, around osteoblasts. This enables various substrates necessary for the construction (formation) of cells to be supplied to osteoblasts through the blood vessels, thereby achieving efficient growth of the osteoblasts. As a result, bone formation is further promoted. For more details on the angiogenesis factor, see the above description of the first embodiment.

Although the second embodiment of the osteogenic treatment device according to the present invention has been described above, the present invention is not limited thereto, as is the case with the first embodiment.

In the second embodiment, a recombinant plasmid obtained by splicing BMP cDNA into an expression plasmid has been described as a representative of nucleic acid containing a base sequence coding for bone morphogenetic factor (BMP), but nucleic acid containing a base sequence coding for BMP to be used in the present invention may be, for example, BMP cDNA (not spliced into an expression plasmid), mRNA of BMP, or one obtained by arbitrarily adding a base(s) to the BMP cDNA or the mRNA of BMP.

In the second embodiment, nucleic acid containing a base sequence coding for bone morphogenetic factor (BMP) has been described as a representative of osteoinductive factors, but an osteoinductive factor to be used in the present invention may be the BMP per se mentioned above.

Hereinbelow, actual examples of the second embodiment of the present invention will be described. In this regard, it is to be noted that Example and Comparative Examples of the first embodiment are marked with a character “A” after the number and Examples and Comparative Examples of the second embodiment are marked with a character “B” after the number to differentiate between first and second embodiments.

### Example 1B

1. Preparation of Recombinant Plasmid

A recombinant plasmid shown in FIG. 1 was obtained by splicing human BMP-2 cDNA (a base sequence coding for human BMP-2) and desired base sequences into an expression plasmid according to a well-known method.

Then, the thus obtained recombinant plasmid was grown in the following manner.

First, the recombinant plasmid was added to 200 μL of a suspension of DH5α (Competent Bacteria) at room temperature to obtain a mixture.

Next, the mixture was added to LB agar medium to carry out cultivation for 12 hours at 37° C.

After the completion of the cultivation, a relatively large colony was selected from colonies grown on the LB agar medium, and was inoculated onto LB agar medium containing Amp (ampicillin) to carry out cultivation for 12 hours at 37° C.

After the completion of the cultivation, the cell membrane of DH5α grown on the LB agar medium containing Amp was broken to obtain a solution, and the recombinant plasmid was purified and separated from the solution.

2. Production of Hydroxyapatite Porous Sintered Body (Base Body)

Hydroxyapatite was synthesized according to a well-known wet synthesis method to obtain a slurry of hydroxyapatite.

The slurry of hydroxyapatite was spray-dried to obtain hydroxyapatite powder having an average particle size of about 15 μm. Thereafter, the hydroxyapatite powder was calcined for 2 hours at 700° C., and was then pulverized by a pulverizer usually used to obtain hydroxyapatite powder having an average particle size of about 12 μm. The hydroxyapatite powder and a 1 wt % aqueous methylcellulose (water-soluble polymer) solution were mixed and were then stirred to obtain a paste of the mixture containing bubbles. It is to be noted that the mixing ratio between the hydroxyapatite powder and the aqueous methylcellulose solution was 5:6 by weight.

The paste of the mixture was poured into a mold, and was then dried at 80° C. so that the water-soluble polymer was turned into a gel. In this way, a molded body was obtained. The molded body was machined so as to have a disk-like shape with a diameter of 10 mm and a thickness of 3 mm (volume: about 0.24 mL) by the use of a processing machine such as a lathe usually used.

The disk-like molded body was sintered for 2 hours at 1,200° C. in the atmosphere to obtain a hydroxyapatite porous sintered body.

The porosity of the hydroxyapatite porous sintered body was 70%. The porosity was measured by the Archimedean method. The value of B/A described above was about 100, and the value of B was about 2.8×10⁵ μm².

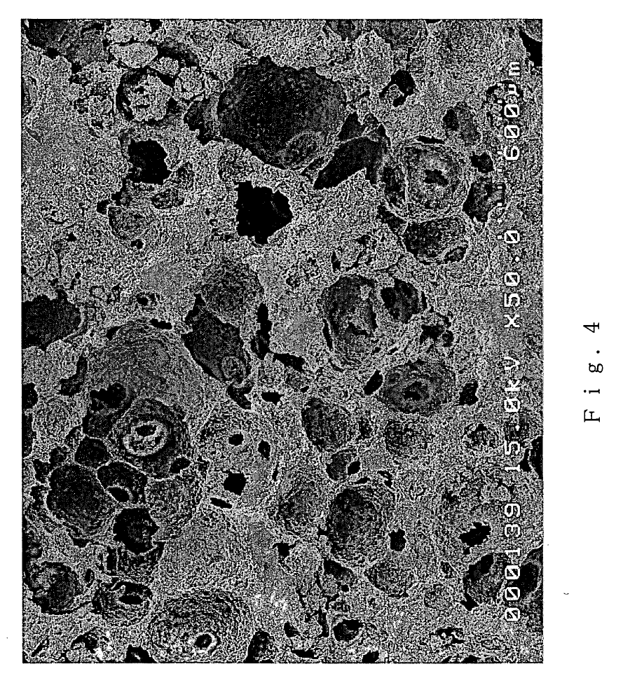

FIG. 4 shows an electron micrograph of the outer surface of the hydroxyapatite porous sintered body, magnified 50 times.

3. Production of Osteogenic Treatment Device

A phosphate buffer containing the recombinant plasmid, a phosphate buffer containing basic Fibroblast Growth Factor (bFGF) as an angiogenesis factor, and a phosphate buffer containing a cationic liposome (“SuperFect”, QIAGEN) as a vector were prepared. The hydroxyapatite porous sintered body was impregnated with these phosphate buffers so as to contain 10 μg of the recombinant plasmid, 1 μg of basic Fibroblast Growth Factor (bFGF), and 40 μg of the cationic liposome. In this way, an osteogenic treatment device was obtained.

### Example 2B

The hydroxyapatite powder obtained in Example 1B, and N,N-dimethyldodecylamineoxide (“AROMOX”, Lion Corporation) as a nonionic surfactant were mixed with a 1 wt % aqueous methylcellulose (water-soluble polymer) solution, and then they were stirred harder than in Example 1B to obtain a paste of the mixture containing bubbles. It is to be noted that the amount of N,N-dimethyldodecylamineoxide contained in the paste of the mixture was 2 wt %.

An osteogenic treatment device was produced in the same manner as in Example 1B except that a hydroxyapatite porous sintered body made using the thus obtained paste of the mixture was used as a base body.

The porosity of the hydroxyapatite porous sintered body (base body) was 85%. The value of B/A was about 3, and the value of B was about 7.1×10⁴ μm².

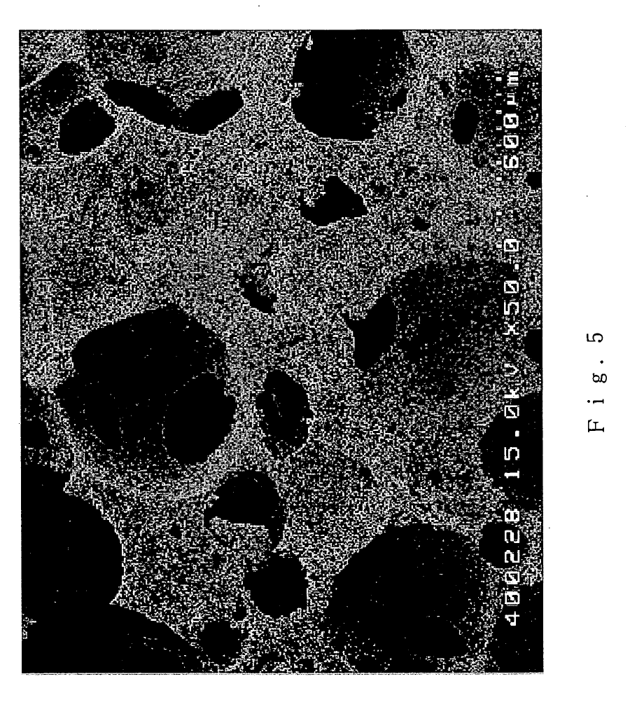

FIG. 5 shows an electron micrograph of the outer surface of the hydroxyapatite porous sintered body, magnified 50 times.

### Comparative Example 1B

A hydroxyapatite porous sintered body was made in the same manner as in Example 2B except that nitrogen gas was blown into the mixture when the mixture was stirred. Then, an osteogenic treatment device was produced in the same manner as in Example 1B except that the thus obtained hydroxyapatite porous sintered body was used as a base body.

The porosity of the hydroxyapatite porous sintered body (base body) was 95%. The value of B/A was about 1.

### Comparative Example 2B

A hydroxyapatite porous sintered body (base body) was made in the same manner as in Example 1B except that a slurry obtained by suspending the hydroxyapatite powder obtained in Example 1B in water was used. Then, an osteoblast treatment device was produced in the same manner as in Example 1B except that the thus obtained hydroxyapatite porous sintered body was used as a base body.

The porosity of the hydroxyapatite porous sintered body (base body) was 30%. The value of B/A was about 160.

Evaluation

1. Evaluation Test

24 domestic rabbits (average body weight: 3.0 kg) were prepared. An operation was performed on each of the domestic rabbits in the following manner.

First, each domestic rabbit was anesthetized with an intravenous injection of 25 mg/kg pentobarbital sodium (“Nembutal”, Abbot Laboratories).

Next, an incision was made in the scalp of the domestic rabbit, and then a flap (width: 2.5 cm, length: 3.0 cm) with a pedicle at the caudal end was elevated.

Next, a 2 to 3 mm of incision was made in the exposed periosteum, and the periosteum having a diameter of about 3 mm was separated from the skull using a periosteum elevator to expose the skull.

Next, a craniotome was set near the midline of the exposed skull, and a through hole was made in the skull by removing it in such a manner that the dura mater was left intact. Thereafter, bleeding was completely stopped. The thickness of the skull was about 3 mm, and the hole made in the skull had a diameter of about 1.2 cm.

Next, the 24 domestic rabbits subjected to craniotomy were divided into 4 groups each containing 6 rabbits. The osteogenic treatment devices of Example 1B, Example 2B, Comparative Example 1B, and Comparative Example 2B were implanted in the domestic rabbits of group 1, group 2, group 3, and group 4, respectively. After implantation, in each of the rabbits the flap was returned to the initial position and then the scalp incision was sutured.

Each of the domestic rabbits subjected to such an operation was bred in an individual cage.

2. Evaluation Result

All the domestic rabbits were killed after the lapse of 9 weeks from the operation with an overdose of the anesthetic described above.

A piece of the skull and skin on the skull were together removed from each domestic rabbit to sample tissue. The tissue was immediately immersed in 10% neutral buffered formalin for fixation, and was then embedded in polyester resin. The tissue embedded in polyester resin was cut and polished to obtain a slice of the tissue having a thickness of 50 μm. Then, cole-HE staining was carried out to obtain a specimen of the tissue.

A bone formation rate was determined using the tissue specimen in the following manner.

Specifically, an image of the tissue specimen was taken with a stereoscopic microscope system SZX-12 provided with a digital camera (DP-12) (Olympus). Next, data of a portion where new bone had been formed was extracted from the image data by digital processing using Photoshop_ver 4.0 (Adobe). Then, the area of the portion where new bone had been formed was measured and digitized by an image analysis method using Scion Image (Scion) to determine a bone formation rate.

It is to be noted that the bone formation rate was determined in an area of 5 mm (measured from the end of the hydroxyapatite porous sintered body in the surface direction thereof, that is, in a direction perpendicular to the thickness direction)×3 mm (that is, the thickness of the hydroxyapatite porous sintered body)+2 mm (measured from the end of the hydroxyapatite porous sintered body on the side of the dura mater in the thickness direction). The bone formation rate was determined in the inside of the hydroxyapatite porous sintered body and on the outer surface of the hydroxyapatite porous sintered body.

The results are shown in Table 2.

TABLE 2

| | Osteogenic Treatment Device | | Evaluation Results | |
|---|---|---|---|---|
| | HAp Porous Sintered Body | | Bone Formation Rate (%) | |
| | B/A | Porosity (%) | Inside | Outer Surface |
| Example 1B | 100 | 70 | 42 ± 10.5 | 25 ± 13.1 |
| Example 2B | 3 | 85 | 40 ± 10.5 | 14 ± 12.1 |
| Com. Ex. 1B | 1 | 95 | 10 ± 10.3 | 40 ± 10.4 |
| Com. Ex. 2B | 160 | 30 | 24 ± 12.4 | 33 ± 11.8 |

HAp: Hydroxyapatite

As shown in Table 2, in the osteogenic treatment devices of Examples 1B and 2B, bone formation occurred more rapidly in the inside of the base body than on the outer surface of the base body.

On the other hand, in the osteogenic treatment devices of Comparative Examples 1B and 2B, bone formation occurred on the outer surface of the base body in preference to the inside of the base body.

From the results, it has become apparent that by appropriately setting the internal shape (the shape of the holes) of the base body, it is possible to promote bone formation along the shape of the base body (that is, along the shape of the site of the implantation).

Further, osteogenic treatment devices were produced in the same manner as in Examples 1B and 2B and Comparative Examples 1B and 2B, respectively, except that in each of the devices a tricalcium phosphate porous sintered body was used as a base body instead of the hydroxyapatite porous sintered body, to carry out the same evaluation test as that described above. The evaluation results were substantially the same as those of Examples 1B and 2B and Comparative Examples 1B and 2B.

Furthermore, osteogenic treatment devices were produced in the same manner as in Examples 1B and 2B except that any one of various human BMPs or nucleic acid containing a base sequence coding for any one of various human BMPs was used as an osteoinductive factor or these osteoinductive factors were arbitrarily combined, to carry out the same evaluation test as that described above. The evaluation results were substantially the same as those of Examples 1B and 2B.

Moreover, osteogenic treatment devices were produced in the same manner as in Examples 1B and 2B except that the angiogenesis factor was changed from basic Fibroblast Growth Factor (bFGF) to Vascular Endothelial Growth Factor (VEGF) or Hepatocyte Growth Factor (HGF) or these angiogenesis factors were arbitrarily combined, to carry out the same evaluation test as that described above. The evaluation results were substantially the same as those of Examples 1B and 2B.

From these results, it can be said that the use of the osteogenic treatment device according to the second embodiment of the present invention enables very rapid bone formation and bone formation along the shape of the site of the implantation, which contributes to rapid osteogenic treatment.

Therefore, the osteogenic treatment device according to the present invention eliminates a necessity to harvest bone to carry out free bone graft. This makes it possible to perform an operation for osteogenic treatment rationally with safety and reliability.

In addition, it is possible to reduce the time required for an operation, the duration of hospitalization, or total medical costs and to improve the quality of treatment and the QOL of patients.

Further, in the present invention, since the angiogenesis factor is used, new blood vessels are actively formed around osteoblasts. This enables efficient growth of osteoblasts and thereby more rapid bone formation.

Furthermore, the use of the vector holding the nucleic acid enhances the efficiency of the uptake of the nucleic acid into the cells involved in bone formation, such as undifferentiated mesenchymal cells, inflammatory cells, and fibroblasts. This also enables more rapid bone formation.

Moreover, the osteogenic treatment device according to the present invention can be easily stored, handled, and shaped into a desired form during an operation.

Finally, it is to be understood that many changes and additions may be made to the embodiments and Examples described above without departing from the scope and spirit of the invention as defined in the following claims.

It is also to be understood that the present application contains subject matter related to the subject matter disclosed in Japanese Patent Application No. 2002-324371 (filed Nov. 7, 2002) and Japanese Patent Application No. 2002-356079 (filed Dec. 6, 2002), the contents of which are entirely incorporated by reference herein.

1. An osteogenic treatment device, comprising:
nucleic acid containing a base sequence coding for bone morphogenetic protein (BMP) and a base sequence derived from an expression plasmid;
an angiogenesis factor;
a non-viral vector for holding the nucleic acid; and
a biocompatible base body;
wherein the angiogenesis factor is mixed with the nucleic acid, in which the mixing ratio between the angiogenesis factor and the nucleic acid is in the range of about 10:1 to 1:100 by weight.

2. The osteogenic treatment device as claimed in claim 1, wherein the base body is constructed from a porous block body having interconnecting holes in which the adjacent holes communicate to each other.

3. The osteogenic treatment device as claimed in claim 2, wherein in a case where the area (average) of boundary parts between the holes adjacent to each other in the base body is defined as A (μm²) and the maximum cross-sectional area (average) of the holes is defined as B (μm²), the value of B/A is in the range of 2 to 150.

4. The osteogenic treatment device as claimed in claim 2, wherein the maximum cross-sectional area (average) B of the holes is in the range of about 7.9×10³ to 1.1×10⁶ μm².

5. The osteogenic treatment device as claimed in claim 2, wherein the porosity of the base body is in the range of 30 to 95%.

6. The osteogenic treatment device as claimed in claim 1, wherein the angiogenesis factor is at least one selected from the group comprising basic Fibroblast Growth Factor (bFGF), Vascular Endothelial Growth Factor (VEGF) and Hepatocyte Growth Factor (HGF).

7. The osteogenic treatment device as claimed in claim 1, wherein the bone morphogenetic protein (BMP) is at least one selected from the group comprising BMP-2, BMP4 and BMP-7.

8. The osteogenic treatment device as claimed in claim 1, wherein the amount of the nucleic acid to be used is in the range of about 1 to 100 μg per 1 mL of the base body.

9. The osteogenic treatment device as claimed in claim 1, wherein the non-viral vector includes a liposome.

10. The osteogenic treatment device as claimed in claim 9, wherein the liposome is a cationic liposome.

11. The osteogenic treatment device as claimed in claim 1, wherein the mixing ratio between the non-viral vector and the nucleic acid is in the range of 1:1 to 20:1 by weight.

12. The osteogenic treatment device as claimed in claim 1, wherein the base body is formed into a block body.

13. The osteogenic treatment device as claimed in claim 1, wherein the base body is porous.

14. The osteogenic treatment device as claimed in claim 13, wherein the porosity of the base body is in the range of 30 to 95%.

15. The osteogenic treatment device as claimed in claim 1, wherein the base body is mainly formed of hydroxyapatite or tricalcium phosphate.

16. An osteogenic treatment device, comprising:
nucleic acid containing a base sequence coding for bone morphogenetic protein (BMP) and a base sequence derived from an expression plasmid;
an angiogenesis factor;
a non-viral vector for holding the nucleic acid; and
a biocompatible base body being constructed from a porous block body having interconnecting holes in which the adjacent holes communicate with each other;
wherein the angiogenesis factor is mixed with the nucleic acid, in which the mixing ratio between the angiogenesis factor and the nucleic acid is in the range of about 10:1 to 1:100 by weight, and wherein in a case where the area (average) of boundary parts between the holes adjacent to each other in the base body is defined as A (μm²) and the maximum cross-sectional area (average) of the holes is defined as B (μm²), the value of B/A is in the range of 2 to 150.
